(19) United States
(12) Patent Application Publication
(10) Pub. No.: US 2017/0335321 A1
(43) Pub. Date: Nov. 23, 2017

(54) MALAT-1, A NON-CODING RNA, IS A TARGET FOR THE REGULATION OF LEARNING AND MEMORY

(71) Applicant: Ibis Biosciences, Inc., Carlsbad, CA (US)

(72) Inventors: Todd P. Michael, Carlsbad, CA (US); Stanley T. Motley, Carlsbad, CA (US)

(21) Appl. No.: 15/522,233

(22) PCT Filed: Nov. 6, 2015
(86) PCT No.: PCT/US2015/059417

§ 371 Date: Apr. 26, 2017

Related U.S. Application Data
(60) Provisional application No. 62/076,352, filed on Nov. 6, 2014.

(51) Int. Cl. C12N 15/113; A61K 48/00

(52) U.S. Cl. CPC C12N 15/113; A61K 48/0075; A61K 48/0016; A61K 48/00

(57) ABSTRACT

Provided herein are methods for improving memory or cognitive function in a subject by administering a composition to the brain of the subject, where the composition comprises: i) a compound that increases expression of MALAT-1 long non-coding RNA, ii) a MALAT-1 long-coding RNA nucleic acid sequence, or iii) at least one MALAT-1 derived piRNA nucleic acid sequence. Also provided herein are methods of screening candidate compounds for their ability to modulate the expression of MALAT-1 long non-coding RNA in brain cells. In certain embodiments, such identified modulators that increase expression are further administered to the brain of a lab animal to determine the impact of such modulators on learning and memory.

### FIELD

Provided herein are methods for improving memory or cognitive function in a subject by administering a composition to the brain of the subject, where the composition comprises: i) a compound that increases expression of MALAT-1 long non-coding RNA, ii) a MALAT-1 long-coding RNA nucleic acid sequence, or iii) at least one MALAT-1 derived piRNA nucleic acid sequence. Also provided herein are methods of screening candidate compounds for their ability to modulate the expression of MALAT-1 long non-coding RNA in brain cells. In certain embodiments, such identified modulators that increase expression are further administered to the brain of a lab animal to determine the impact of such modulators on learning and memory.

### BACKGROUND

Neurological functions and pathologies and resulting properties and phenotypes (e.g., behavior, memory, disease, etc.) are fundamentally important aspects of animal (e.g., human) biology, health, and well-being. Yet the underlying molecular and cellular biology is poorly understood. In view of this, there is a dearth of pharmaceutical or research tools for altering these properties and phenotypes at the molecular level and in a specific manner.

### SUMMARY

Provided herein are methods for improving memory or cognitive function in a subject by administering a composition to the brain of the subject, where the composition comprises: i) a compound that increases expression of MALAT-1 long non-coding RNA, ii) a MALAT-1 long-coding RNA nucleic acid sequence, or iii) at least one MALAT-1 derived piRNA nucleic acid sequence (e.g., one or more of SEQ ID NOS:5-22 or sequences with 90-99% sequence identity with SEQ ID NOS:5-22). Also provided herein are methods of screening candidate compounds for their ability to modulate the expression of MALAT-1 long non-coding RNA in brain cells. In certain embodiments, such identified modulators that increase expression are further administered to the brain of a lab animal to determine the impact of such modulators on learning and memory.

In some embodiments, provided herein are methods of for improving memory or cognitive function in a subject, comprising: administering to the subject (e.g., a human subject) a therapeutically effective dose of a composition that increases the expression of metastasis-associated lung adenocarcinoma transcript 1 (MALAT-1) long non-coding RNA in a cell of the nervous system of the subject, wherein the administering is performed intracranially or wherein the administering is performed in a blood vessel that directly supplies blood to the brain of the subject; wherein the composition comprises at least one of the following: i) a compound (e.g., identified by the screening methods described herein) that increases expression of MALAT-1 long non-coding RNA in the cell, ii) a first nucleic acid sequence comprising the MALAT-1 long-coding RNA or a first expression vector encoding the first nucleic acid sequence; or iii) a second nucleic acid sequence encoding at least one MALAT-1 derived piRNA sequence (e.g., one or more of SEQ ID NOS:5-22) or a second expression vector encoding the second nucleic acid sequence; and wherein at least one attribute of the memory or cognitive function is improved.

In certain embodiments, the subject has a disorder in which diminished declarative memory is a symptom. In other embodiments, the molecule is KCl or a DNA methyltransferase (DNMT) inhibitor. In further embodiments, the DNMT inhibitor comprises RG108. In additional embodiments, the first and/or second expression vector comprises an adeno-associated virus (AAV), adenovirus, herpes simplex virus, lentivirus, or a DNA plasmid. In additional embodiments, the composition is administered to the subject by intracranial delivery through an intracranial access device. In some embodiments, the method further comprises the step of: implanting a pump outside the brain of the subject, wherein the pump is coupled to the proximal end of the intracranial access device. In particular embodiments, the intracranial access device comprises an intracranial catheter.

In other embodiments, the first and/or second nucleic acid molecule comprises a chemical modification that improves one or more or all of nuclease stability, decreased likelihood of triggering an innate immune response, lowering incidence of off-target effects, and improved pharmacodynamics relative to a non-modified nucleic acid. In further embodiments, the at least one chemical modification selected from the group consisting of: phosphorothioate, boranophosphate, 4′-thio-ribose, locked nucleic acid, 2′-O-(2′-methoxyethyl), 2′-O-methyl, 2′-fluoro, 2′-deoxy-2′-fluoro-b-D-arabinonucleic acid, Morpholino nucleic acid analog, and Peptide nucleic acid analog. In some embodiments, the first and/or second nucleic acid sequence is attached to, or inside of, a nanoparticle configured to cross the blood-brain barrier. In certain embodiments, the nanoparticle comprises a liposome. In additional embodiments, the composition is delivered to the nucleus basalis of Meynert, the cerebral cortex, or the hippocampus.

In some embodiments, the subject has Alzheimer's disease and/or age related memory decline. In further embodiments, the subject has a memory impairment. In additional embodiments, the memory impairment is selected from the group consisting of: toxicant exposure, brain injury, age-associated memory impairment, mild cognitive impairment, epilepsy, mental retardation, and dementia resulting from a disease. In other embodiments, the disease that results in dementia is selected from the group consisting of: Parkinson's disease, Alzheimer's disease, AIDS, head trauma, Huntington's disease, Pick's disease, Creutzfeldt-Jakob disease, post cardiac surgery, Downs Syndrome, Anterior Communicating Artery Syndrome, and symptoms of stroke. In particular embodiments, the subject has normal memory function that is desired to be enhanced. In some embodiments, the administering is performed in a blood vessel that directly supplies blood to the brain of the subject.

The present disclosure provides methods for screening and identifying compounds that modulate the expression of MALAT-1 long non-coding RNA in brain cells comprising: a) contacting brain cells with a candidate agent, and b) detecting the expression level of said MALAT-1 long non-coding RNA (e.g., all or a portion of SEQ ID NO:1) and/or a MALAT-1 derived piRNA (e.g., one or more of SEQ ID NOS:5-22)), wherein an increase or decrease in said expression level indicates that said candidate agent is a modulator of MALAT-1 long non-coding RNA in brain cells. In certain embodiments, the identified modulator increases the expression level of MALAT-1 RNA, and the method further comprises administering the identified modulator of MALAT-1 long non-coding RNA to the brain of a lab animal, and determining the impact of said modulator on memory or learning of the lab animal. In further embodiments, the modulator is identified as increasing memory and/or learning in said animal. In particular embodiments, the brain cells are neurons or glial cells.

In certain embodiments, the present disclosure provides methods for treating alcoholism in a subject, comprising: administering to the subject a therapeutically effective dose of a composition that comprises a MALAT-1 antisense (e.g., SEQ ID NOs: 2-4, or sequences with 90-99% identity with SEQ ID NOs:2-4), wherein the administering is performed intracranially or wherein the administering is performed in a blood vessel that directly supplies blood to the brain of the subject; and wherein at least one attribute of alcoholism in the subject is improved.

### DESCRIPTION OF DRAWINGS

FIG. 1 shows a schematic of the threat recognition behavior assay used in Example 1.

FIG. 2 shows that mice subject to fear conditioning training, exhibit increased freezing when reintroduced to the training context.

FIG. 3 shows that after mouse training (context+shock) there is a significant increase in MALAT1 expression at 2 hours compared to control (context alone or naïve) consistent with a role in behavior based learning.

FIG. 4 shows that the infusion of the MALAT1 anti-sense oligonucleotide (ASO) leads to decreased freezing in the fear conditioning model at 24 hours post training, which suggests that the mouse is not consolidating the memory as effectively without MALAT1.

FIG. 5 shows that there was no significant change in gene expression of the immediate early genes (IEGs) between mice treated with MALAT1 ASO and the control mice. In this figure M=malat1 ASO treated and P=PBS controls.

FIG. 6 shows that there was a significant increase in small RNA abundance in MALAT1 ASO treated mice.

FIG. 7 shows that DNA methylation was increased in MALAT1 ASO treated mice compared to controls.

### DEFINITIONS

As used herein, the terms “host,” “subject” and “patient” refer to any animal, including but not limited to, human and non-human animals (e.g., dogs, cats, cows, horses, sheep, poultry, fish, crustaceans, etc.) that is studied, analyzed, tested, diagnosed or treated. As used herein, the terms “host,” “subject” and “patient” are used interchangeably, unless indicated otherwise.

As used herein, the term “effective amount” refers to the amount of a composition (e.g., a synthetic MALAT-1 derived piRNA) sufficient to effect beneficial or desired results. An effective amount can be administered in one or more administrations, applications or dosages and is not intended to be limited to a particular formulation or administration route.

As used herein, the terms “administration” and “administering” refer to the act of giving a drug, prodrug, or other agent, or therapeutic treatment (e.g., compositions of the present invention) to a subject (e.g., a subject or in vivo, in vitro, or ex vivo cells, tissues, and organs). Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal), ear, rectal, vaginal, by injection (e.g., intravenously, subcutaneously, intratumorally, intraperitoneally, etc.) and the like.

As used herein, the terms “co-administration” and “co-administering” refer to the administration of at least two agent(s) (e.g., multiple synthetic MALAT-1 derived piRNAs or a piRNA or anti-piRNA molecule and another therapeutic) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, the term “treatment” or grammatical equivalents encompasses the improvement and/or reversal of the symptoms of disease (e.g., neurodegenerative disease) or condition. A compound which causes an improvement in any parameter associated with disease when used in the screening methods of the instant invention may thereby be identified as a therapeutic compound. The term “treatment” refers to both therapeutic treatment and prophylactic or preventative measures. For example, those who may benefit from treatment with compositions and methods of the present invention include those already with a disease and/or disorder (e.g., neurodegenerative disease) as well as those in which a disease and/or disorder is to be prevented (e.g., using a prophylactic treatment).

The term “compound” refers to any chemical entity, pharmaceutical, drug, and the like that can be used to treat or prevent a disease, illness, sickness, or disorder of bodily function. Compounds comprise both known and potential therapeutic compounds. A compound can be determined to be therapeutic by screening using screening methods. A “known therapeutic compound” refers to a therapeutic compound that has been shown (e.g., through animal trials or prior experience with administration to humans) to be effective in such treatment. In other words, a known therapeutic compound is not limited to a compound efficacious in the treatment of disease (e.g., neurodegenerative disease).

As used herein, the term “pharmaceutical composition” refers to the combination of an active agent (e.g., a MALAT-1 derived piRNA) with a carrier, inert or active, making the composition especially suitable for diagnostic or therapeutic use in vitro, in vivo or ex vivo.

As used herein, the term “nucleic acid molecule” refers to any nucleic acid containing molecule, including but not limited to, DNA or RNA (e.g., MALAT-1 derived piRNA). The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxy-aminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

As used herein, the terms “gene expression” and “expression” refer to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refer to regulation that increases and/or enhances the production of gene expression products (e.g., RNA or protein), while “down-regulation” or “repression” refer to regulation that decrease production.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is present in a form or setting that is different from that in which it is found in nature. In contrast, non-isolated nucleic acids are nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form.

The term “synthetic” when used in reference to nucleic acid molecules (e.g., piRNA) refers to non-natural molecules made directly (e.g., in a laboratory) or indirectly (e.g., from expression in a cell of a construct made in a laboratory) by mankind

### DETAILED DESCRIPTION

Provided herein are methods for improving memory or cognitive function in a subject by administering a composition to the brain of the subject, where the composition comprises: i) a compound that increases expression of MALAT-1 long non-coding RNA, ii) a MALAT-1 long-coding RNA nucleic acid sequence, or iii) at least one MALAT-1 derived piRNA nucleic acid sequence. Also provided herein are methods of screening candidate compounds for their ability to modulate the expression of MALAT-1 long non-coding RNA in brain cells. In certain embodiments, such identified modulators that increase expression are further administered to the brain of a lab animal to determine the impact of such modulators on learning and memory.

MALAT-1 is a non-coding RNA that has been implicated as a regulator of metastasis in lung cancer (Cancer Res. 2013 Feb. 1; 73(3):1180-9, herein incorporated by reference) is upregulated in the brains of alcoholics (Kryger, et al., Alchol, 46, 629-634, 2012, herein incorporated by reference) and enhances the motility of lung adenocarcinoma cells (Tano et al., FEBS Letters, 584:4575-4580, 2010, herein incorporated by reference). Work conducted during development of embodiments of the present disclosure indicated that MALAT1 RNA was highly expressed in the hippocampus of mouse brains as a small RNA identified by deep sequence analysis of the RNA from cultures of mouse hippocampus cells. Expression is increased upon treatment with KCI (a mock learning event for neuronal cell cultures) and RG108 (a DNMT inhibitor) and the effect is additive with the highest levels of expression observed with treatment with both KCI and RG108. This is consistent with MALAT-1 being involved in learning and memory formation. When mouse cortical neuronal cultures are stimulated with KCI with or without the presence of RG108, expression of MALAT-1 is induced. Impressively, MALAT-1 expression in the CA1 region of the brain is induced after the fear conditioning/learning paradigm. This data implicates a significant involvement of MALAT-1 in the process of learning and memory.

In some embodiments, provided herein are synthetic MALAT-1 derived piRNA molecules (e.g., SEQ ID NOS: 5-22 or sequences with 90-99% sequence identity with SEQ ID NOS:5-22)). In some embodiments, the piRNA molecules comprise chemical modification to improve nuclease stability, decrease the likelihood of triggering an innate immune response, lower the incidence of off-target effects, and/or improve pharmacodynamics relative to non-modified molecules so as to increase potency and specificity. In some embodiments, the molecules are loaded onto nanoparticles, providing a stabilizing effect (e.g., protecting against nuclease degradation). These effects are particularly important for nucleic acids intended to treat the brain, where the delivery challenges limit the amount of active nucleic acid drug that will reach the target cells. Exemplary chemical modifications of nucleotides (e.g., modifications of the sugars) in the synthetic piRNA molecules that find use in some embodiments of the technology include the following: phosphorothioate, boranophosphate, 4′-thio-ribose, locked nucleic acid, 2′-O-(2′-methoxyethyl), 2′-O-methyl, 2′-fluoro, 2′-deoxy-2′-fluoro-b-D-arabinonucleic acid, Morpholino nucleic acid analog, and Peptide nucleic acid analog. Additional modification used with antisense oligonucleotides may be employed (see e.g., US Pat. Publ. Nos. 2012/0202874 and 2012/0149755, herein incorporated by reference in their entireties).

Delivery of the nucleic acids sequences described herein (e.g., synthetic MALAT-1 derived piRNA molecules or MALAT-1 antisense oligonucleotides) may be accomplished by any desired method. In some embodiments, molecules are delivered intrathecally, intracranially, or in a blood vessel that leads directly to the brain. In some embodiments, a Medtronic infusion system employing an implantable, battery-powered drug-infusion pump is used to deliver molecules to the striatum (Dickinson et al., Neuro. Oncol. 12:928-940 (2010); Sah and Aronin, J. Clin. Invest. 121: 500-507 (2011)). In some embodiments, intranasal delivery is used. In some embodiments, nucleic acids are delivered by nanoparticles. For example, particles comprising an iron-oxide core coated with chitsan may be used (see e.g., Veiseh et al., Adv. Drug Deliv. Rev., 8:582 (2011)). Chitosan is a transcytosing molecule that is able to cross the blood brain barrier. In some embodiments, the particles are associated with a call-penetrating peptide to facilitate delivery of the nucleic into cells. In some embodiments, endogenous nanoparticles (e.g., high-density lipoproteins) are used to deliver molecules across the blood brain barrier.

In some embodiments, compounds and oligonucleotides are delivered/administered directly to the brain, for example, through intrathecal injections (e.g., in humans), ICV (e.g., in mice, rats and humans), intracerebrocentricular injection (a type of injection into the ventricular system of the brain), or by direct injection into the specific area of the brain to be interrogated.

In some embodiments, Malat1 expression is upregulated by introducing synthetic oligonucleotides (e.g., similar to piRNAs or microRNAs) that alter the methylation state of the Malat1 promoter, thus increasing transcription.

In other embodiments, since Malat1 is a long non-coding RNA, and they have been shown to have enhancer like functions on gene expression, enhancers are utilized to increase the actual expression of Malat1 (Ørom et al. Cell. 2010 Oct. 1; 143(1):46-58; herein incorporated by reference in its entirety).

In certain embodiments, gene therapy, utilizing zinc finger recombinase fusion proteins, is used to site-specifically exchange the promoter of Malat1 with a promoter that has constitutive or higher level expression. In some embodiments, the Tet promoter, for example, is used, and after recombining in the targeted cells, the gene is turned on by feeding tetracycline to the subject. One exemplary alternative of this approach is to introduce the Malat1 gene behind the native promoter with the desired level of expression.

In some embodiments, the expression of negative regulators of Malat1 are reduced and/or inhibited, thereby increasing the expression of Malat1. Similarly, in certain embodiments, the degradation of Malat1 is reduced by inhibiting the degradation pathways.

Experiments conducted during development of embodiments described herein have demonstrated that stimulation of cells with KCl (which mimics a learning event) increases the expression of Malat1. Therefore, in some embodiments, Malat1 expression is stimulated with a strong learning event. In other embodiments, deep brain stimulation (e.g., by implanting electrodes or by external transcranial direct current electrical stimulation), which has been shown to increase learning, is used to stimulate the production of Malat1.

In some embodiments, exosomes loaded with Malat1 RNA that are carrying external markers that direct the exosomes to the desired region of the brain (or any organ) are administered (e.g., injected peripherally).

In certain embodiments in which the composition is delivered across the blood-brain barrier, the composition includes, for example, a liposome as described, for example, in U.S. Pat. No. 6,372,250 (Pardridge), and a pharmaceutically acceptable carrier. Preferably the liposome is a receptor-specific liposome, wherein the receptor-specific liposome includes: a liposome having an exterior surface and an internal compartment; an artificial adeno-associated virus (AAV) vector located within the internal compartment of the liposome; one or more blood-brain barrier and brain cell membrane targeting agents; and one or more conjugation agents (e.g., polyethylene glycol (PEG) strands), wherein each targeting agent is connected to the exterior surface of the liposome via at least one of the conjugation agents. Receptor-specific liposomes including an artificial adeno-associated virus (AAV) vector located within the internal compartment of the liposome can be prepared by the general methods described in U.S. Pat. No. 6,372,250 (Pardridge), except that the artificial adeno-associated virus (AAV) vector is used instead of the plasmid DNA.

The present disclosure provides methods for screening and identifying compounds that modulate the expression of MALAT-1 long non-coding RNA in brain cells comprising: a) contacting brain cells with a candidate agent, and b) detecting the expression level of said MALAT-1 long non-coding RNA and/or a MALAT-1 derived piRNA, wherein an increase or decrease in said expression level indicates that said candidate agent is a modulator of MALAT-1 long non-coding RNA in brain cells. In certain embodiments, the identified modulator increases the expression level of MALAT-1 RNA, and the method further comprises administering the identified modulator of MALAT-1 long non-coding RNA to the brain of a lab animal, and determining the impact of said modulator on memory or learning of the lab animal. In further embodiments, the modulator is identified as increasing memory and/or learning in said animal. In particular embodiments, the brain cells are neurons or glial cells.

The candidate agents (i.e., test compounds) of the present disclosure can be obtained, for example, using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 (1994)); spatially addressable parallel solid phase or solution phase libraries; synthetic library methods requiring deconvolution; the ‘one-bead one-compound’ library method; and synthetic library methods using affinity chromatography selection. The biological library and peptoid library approaches are generally preferred for use with peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 (1993); Erb et al., Proc. Nad. Acad. Sci. USA 91:11422 (1994); Zuckermann et al., J. Med. Chem. 37:2678 (1994); Cho et al., Science 261:1303 (1993); Carrell et al., Angew. Chem. Int. Ed. Engl. 33.2059 (1994); Carell et al., Angew. Chem. Int. Ed. Engl. 33:2061 (1994); and Gallop et al., J. Med. Chem. 37:1233 (1994). Libraries of compounds may be presented in solution (e.g., Houghten, Biotechniques 13:412-421 (1992)), or on beads (Lam, Nature 354:82-84 (1991)), chips (Fodor, Nature 364:555-556 (1993)), bacteria or spores (U.S. Pat. No. 5,223,409; herein incorporated by reference), plasmids (Cull et al., Proc. Nad. Acad. Sci. USA 89:18651869 (1992)) or on phage (Scott and Smith, Science 249:386-390 (1990); Devlin Science 249:404-406 (1990); Cwirla et al., Proc. Natl. Acad. Sci. 87:6378-6382 (1990); Felici, J. Mol. Biol. 222:301 (1991)).

Methods for screening for MALAT1-targeting compounds may be performed by cell-based assays or cell free assays.

In some embodiments, for cell free assays, a full length Malat1 RNA is provided along with PIWI proteins, synthetic piRNAs, and plasmid DNA carrying sequences to be interrogated. Following incubation, the level of methylation on the plasmid DNA is determined by direct sequencing, or if an expression construct is utilized, the level of in vitro expression from a promoter is determined by incubating with the associated RNA polymerase and monitoring the levels of RNA transcription.

In some embodiments, for cell-based assays, primary cells from cortex or hippocampus or transformed neuronal cell lines are used to identify regions of import for learning and memory. Other cell lines from appropriate tissues are used to investigate performance and effects as required. Cells are plated and grown to near confluancy. The cells are then transfected with the synthetic oligos. After a period of time (e.g., 2 hours-2 weeks), cells are harvested, and RNA and DNA is extracted. The RNA is used in, for example, RNA sequencing experiments to identify changes in gene expression and also small RNA production. The DNA is used to identify changes in DNA methylation with methylation DNA-seq (e.g., the current state of the art is conversion of the DNA with bisulfite chemistry, followed by single nucleotide sequencing on the Illumina HiSeq 2500). Alternatively, DNA methylation is identified directly with the Pacific Biosciences DNA sequencing technology, or similar technology, that directly identifies methylation of specific bases.

### EXAMPLES

### Example 1

### MALAT-1 is Involved in Regulation of Learning and Memory

This Examples describes the identification and characterization of the non-coding RNA MALAT-1 as involved in the regulation of learning and memory.

Male C57BL/6J mice (Jackson Laboratories) of approximately 9-12 weeks of age were used for the experiments. Animals were pair housed upon arrival and food and water were available ad libitum. Animals were given at least one week to habituate to the colony before inclusion in experiments. All protocols complied with the National Institute of Health Guide for the Care and Use of Laboratory Animals and were approved by the University of Alabama at Birmingham Animal Care Committee. All animals were handled for 4 days prior to threat recognition learning.

Mice were placed in the training chamber and given 2 minutes to explore the novel context. After 2 min, mice received 3 electric footshocks (0.7 mA, 2 sec) administered 1 minute apart, with an additional minute allowed for exploration before removal from the chamber. Animals were euthanized via rapid decapitation 1 hour following training. Brains were submerged in oxygenated (95%/5% O2/CO2) ice-cold cutting solution (125 mM NaCl, 3 mM KCl, 1.25 mM NaH2PO4, 25 mM NaHCO3, 0.5 mM CaCl2, 7 mMMgCl2, 10 mM glucose, 0.6 mM ascorbate) immediately after rapid decapitation and during gross dissection of hippocampi, cortices, and cerebella.

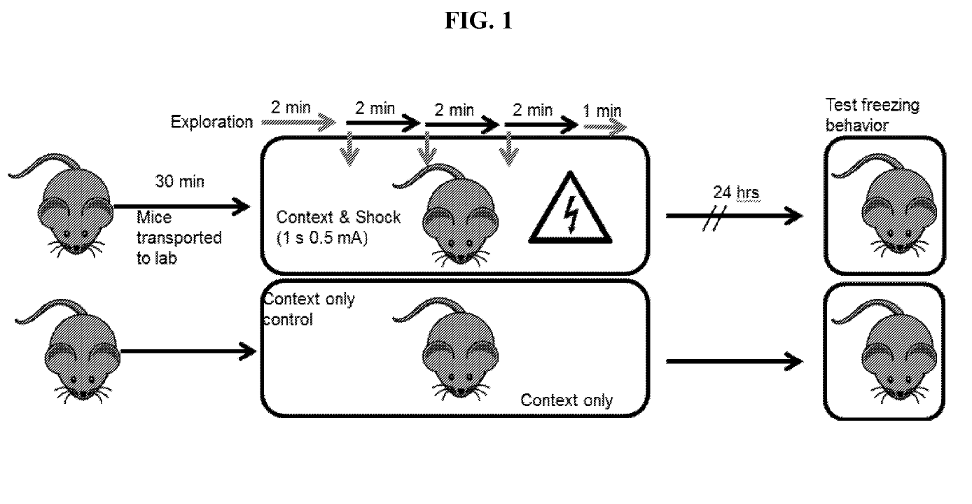

Percent freezing is a measure of how well the animals remember the experience (mild foot shock) that they received in a particular environment. Naïve animals are placed in the new “context” and experience a series of 3 foot shocks (1 s, 0.5 mA) with 2 m in between shocks. 24 hours later the animals are returned to the environment and the amount of time they stay still is measured. In this case, context plus shock causes a significant increase in freezing as compared to context alone. A schematic of the threat recognition behavior assay is shown in FIG. 1.

Young male C57B6 mice were subjected to a standard fear recognition training protocol. This includes putting the mice into a novel environment for 2 m, followed by 3 foot-shocks (12, 0.5 mA) at 2 minute intervals. The control mice were also put into the novel environment but did not receive the foot shocks. All mice are then returned to their home cages for 24 hours. The mice are then again put into the novel environment and the percent freezing is measured. Freezing is a measure of how well the animals remember the fear inducing experience (foot shock) as compared to the animals that experienced the context alone.

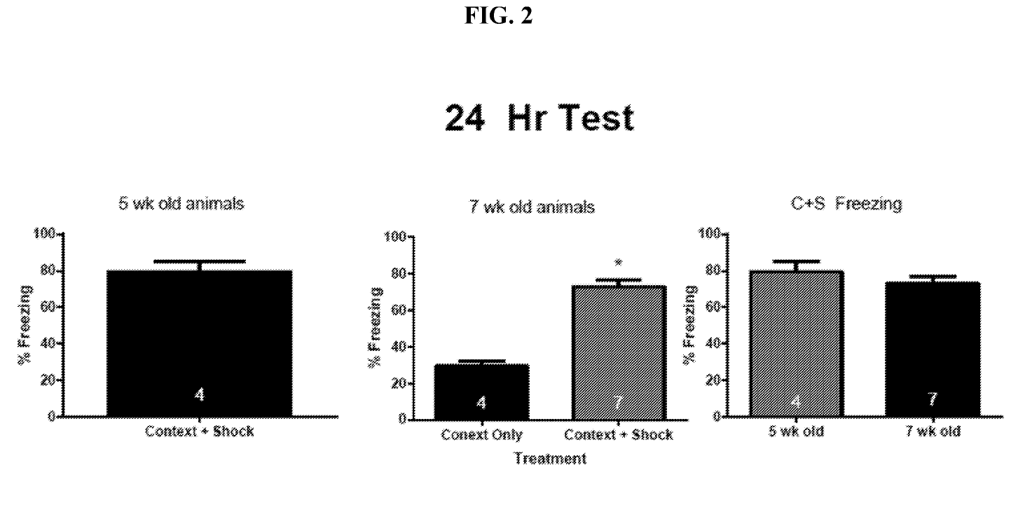

As shown in FIG. 2, mice subject to fear conditioning training, exhibit increased freezing when reintroduced to the training context. It is noted that, the use of the phrase “threat recognition behavior training” is equal to “fear conditioning training”. “Fear conditioning” is the term used most often in the scientific literature and “threat recognition” is used in the military context.

In additional work, the animals are put through the same training protocol as described above except that the animals are sacrificed at various time points post training (30 m, 2 h and 24 h). The hippocampus was dissected with oxygenated cutting solution (high in Mg²⁺ and low in Ca²⁺ to prevent excitotoxicity). Subsequently, RNA was extracted from the hippocampus (using the Qiagen miRNeasy kit). An aliquot of RNA was used for in-house qPCR to validate Malat-1 expression.

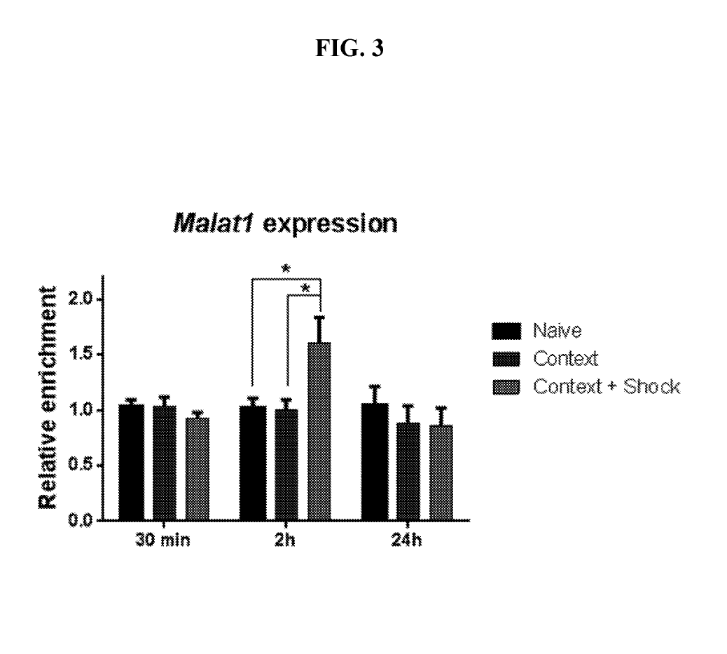

FIG. 3 shows that after training (context+shock) there is a significant increase in MALAT1 expression at 2 hours compared to control (context alone or naïve) consistent with a role in behavior based learning. 24 hours after training, MALAT1 expression returns to that of the controls. Together these results show that an activity based learning event causes a time-dependent increase in MALAT1, consistent with a role in learning and memory consolidation.

In further work, stereotaxic surgery for anti-sense oligonucleotide (ASO) delivery was conducted. Mice were anesthetized with isoflurane and secured in a Kopf stereotaxic apparatus. ASOs (300 ug at 60 ug/ul) were delivered into the ICV with the following coordinates: (AP −0.2; ML −1.0; DV −2.4) at a rate of 1 ul/min, with 2 weeks allowed for recovery.

MALAT1 ASO #1 (SEQ ID NO: 2) was injected by IntraCerebroVentricular bolus (ICVB) injection (300 ug, 180 ug or PBS control). After 2 weeks of post-surgical recovery, the animals were put through the Fear Conditioning training as described above, and 24 hours later tested for fear response.

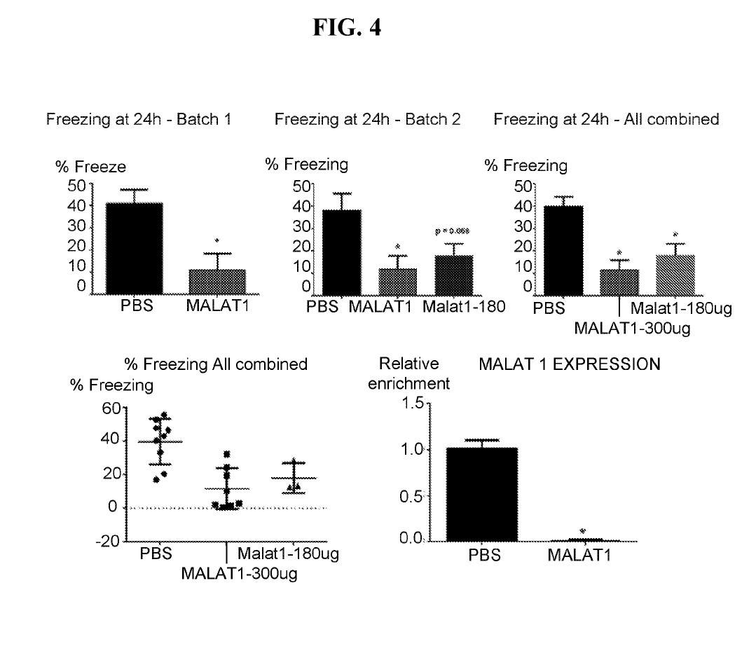

As shown in FIG. 4, infusion of the MALAT1 ASO leads to decreased freezing in the fear conditioning model at 24 hours post training, which suggests that the mouse is not consolidating the memory as effectively without MALAT1. The fact that the mouse does not learn the shock as well without MALAT1 is consistent with MALAT1 increasing in expression 2 hours after training. The experiment has been repeated three times (labeled as batches in the figure); only batches one, two and the combined results are shown. This result is very significant because loss of MALAT1 shows a clear impact on learning. There is also a dose response with 300 ug resulting in a stronger response than the 180 ug dose. The molecular knockdown (qPCR) of the MALAT1 transcript is confirmed in the bottom right graph of FIG. 4 (300 ug dose).

In additional work, the expression of Immediate Early Genes (IEGs) in mice treated with MALAT1 ASO's was monitored. MALAT1 ASO #1 (SEQ ID NO: 2) was injected by IntraCerebroVentricular bolus (ICVB) injection (300 ug v. PBS control). After 2 weeks of post-surgical recovery, the animals were put through the Fear Conditioning training as described above. One hour after training the animals were sacrificed, the hippocampus was removed, RNA was extracted and used for quantitation of gene expression via qPCR.

RNA Extraction and qRT-PCR was performed as follows. Using the miRNeasy mini kit (Qiagen), total RNA was extracted following the manufacturer's guidelines with the additional RNase-free DNase (Qiagen) treatment step, and eluted in 104 uL RNase-free water. For in vivo studies, the right hippocampus was processed. For in vitro studies, purified RNA was pooled across three wells of a 12-well plate to increase yields of RNA necessary for sequencing. RNA concentrations were determined spectrophotometrically using the NanoDrop 200c (Thermo Scientific). 300 ng of RNA was then reverse transcribed into cDNA by using oligo-(dT) and random hexamer primers in the iScript cDNA synthesis kit (Bio-Rad). Quantitative reverse transcriptase PCR (qRT-PCR) was carried out using a CFX96 touch real-time PCR detection system (Bio-Rad) with either SSO Advanced Universal SYBR Green Supermix (Bio-Rad) and 500 nM of intron-spanning primers (Table 1) or Taqman Fast Advanced Master Mix and Taqman gene expression assays (Life Technologies) (Table 2).

TABLE 1
Primers used.

| | Sense primer | Antisense primer |
|---|---|---|
| Gene | (5′->3′) | (5′->3′) |
| Hprt | GGAGTCCTGTTGATGTTGCC | GGGACGCAGCAACTGACATTTCTA |
| | AGTA (SEQ ID NO: 23) | (SEQ ID NO: 24) |
| Fos | AATGGTGAAGACCGTGTCAG | TTGATCTGTCTCCGCTTGGAGTGT |
| | GA (SEQ ID NO: 25) | (SEQ ID NO: 26) |
| Arc | ACGATCTGGCTTCCTCATTC | AGGTTCCCTCAGCATCTCTGCTTT |
| | TGCT (SEQ ID NO: 27) | (SEQ ID NO: 28) |
| Egr1 | AGCGCCTTCAATCCTCAAG | TTTGGCTGGGATAACTCGTC |
| | (SEQ ID NO: 29) | (SEQ ID NO: 30) |

TABLE 2
Taqman gene expression assays used.

| Gene | Product Size | | Catalog Number |
|---|---|---|---|
| Hprt | 65 | bp | Mm00446968_m1 |
| Malat-1 | 124 | bp | Mm03958568_s1 |

PCR amplifications were performed in triplicate with the following cycling conditions: 95° C. for 30 second, followed by 40 cycles of 95° C. for 10 second and 60° C. for 30 seconds, followed by real-time melt analysis to verify product specificity (SYBR) or 50° C. for 2 min, followed by 95° C. for 20 seconds, followed by 40 cycles of 95° C. for 3 seconds and 60° C. for 30 seconds (Taqman). Differential gene expression between samples was determined by the comparative Ct (ΔΔCt) method using hypoxanthine-guanine phosphoribosyltransferase (Hprt) as an internal control.

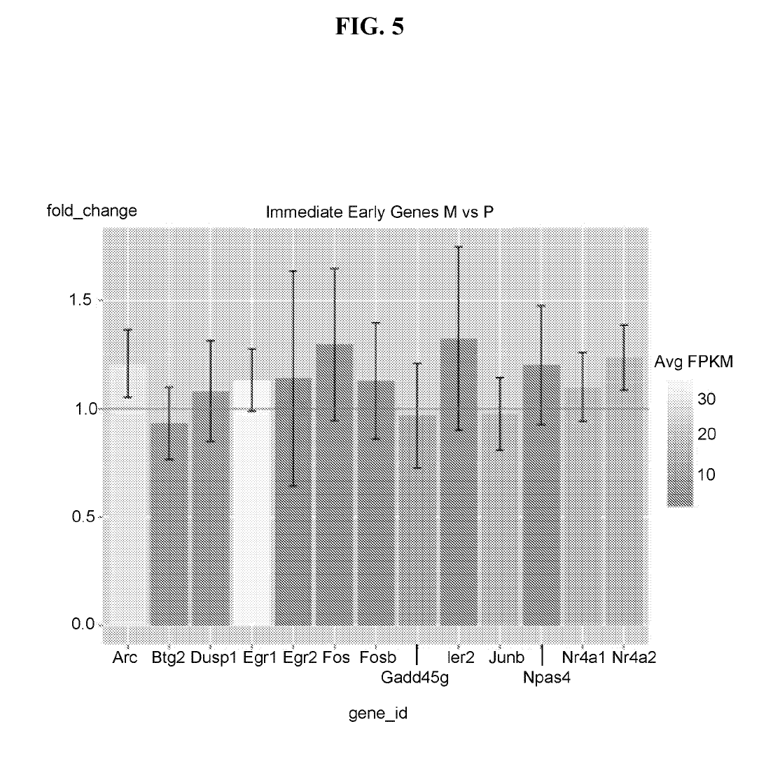

The expression of the Immediate Early Genes (IEGs) has been shown to increase after a learning event. This Example monitored the expression of the canonical IEGs Arc, Btg2, Dusp1, Egr1, Egr2, Fos, Fosb, Gadd45g, ler2, Junb, Npas4, Nr4a1 and Nr4a2 for their expression in mice treated with the MALAT1 ASO. As shown in FIG. 5, it was found that there was no significant change in gene expression of these IEGs between mice treated with MALAT1 ASO and the control mice. Therefore the reduced learning observed in the MALAT1 ASO treated mice is not due to altered expression of the IEG genes. These results suggest that MALAT1 is acting to increase learning through a novel mechanism that has yet to be described in the literature.

In further work, the expression levels of small RNAs was examined in MALAT1 anti-sense oligo (ASO) treated mice. Exemplary ASO (SEQ ID NOS: 2-4) from MALAT1 (SEQ ID NO:1) are provided in Table 3.

TABLE 3
Exemplary anti-sense oligonucleotides

| Isis | | Ext. | | | | | Chemistry |
|---|---|---|---|---|---|---|---|
| No | Targets | Coeff. | MW | Length | Sequence | Chemistry | Notation |
| 626112 | metastasis | 194.44 | 7152.86 | 20 | GCCAGGCTGGTTATGACTCA | 5-10-5 | Ges mCeo mCeo |
| | associated | | | | | MOE | Aeo Geo Gds |
| | lung adeno- | | | | | gapmer | mCds Tds Gds |
| | carcinoma | | | | | w/mixed | Gds Tds Tds |
| | transcript | | | | | backbone | Ads Tds Gds |
| | 1 (non- | | | | | [AGTmC] | Aeo mCeo Tes |
| | coding RNA) | | | | | | mCes Ae |
| | Mouse | | | | | | |
| | (36092) | | | | | | |
| 655125 | metastasis | 201.24 | 7188.86 | 20 | CGGTGCAAGGCTTAGGAATT | 5-10-5 | mCes Geo Geo |
| | associated | | | | | MOE | Teo Geo mCds |
| | lung adeno- | | | | | gapmer | Ads Ads Gds |
| | carcinoma | | | | | w/mixed | Gds mCds Tds |
| | transcript | | | | | backbone | Tds Ads Gds |
| | 1 (non- | | | | | [AGTmC] | Geo Aeo Aes |
| | coding RNA) | | | | | | Tes Te |
| | Mouse | | | | | | |
| | (36092) | | | | | | |
| 702141 | metastasis | 194.66 | 7193.94 | 20 | GGGTCAGCTGCCAATGCTAG | 5-10-5 | Ges Geo Geo |
| | associated | | | | | MOE | Teo mCes Ads |
| | lung adeno- | | | | | gapmer | Gds mCds Tds |
| | carcinoma | | | | | w/mixed | Gds mCds mCds |
| | transcript | | | | | backbone | Ads Ads Tds |
| | 1 (non- | | | | | [AGTmC] | Geo mCeo Tes |
| | coding RNA) | | | | | | Aes Ge |
| | Mouse | | | | | | |
| | (36092) | | | | | | |

RNA from MALAT1 ASO treated and control mice was isolated, and subjected to RNA-seq and small RNA sequencing. The paired-end mRNA reads were trimmed for adapter contamination and low quality sequence. These were then aligned with Tophat2 to the mouse genome GRCm38.p2 using the annotated transcriptome to guide alignments crossing known splice junctions. The alignments are then processed with Cufflinks to detect expressed transcripts. All of the resultant GTF files and the reference annotation are pooled together with cuffmerge to build the consensus transcript assembly for which abundances are calculated. The small RNA samples yield 1×67 bp reads, which were trimmed for both adapter sequence and quality with trimmed reads shorter than 15 bp being discarded. The trimmed reads were aligned to the reference genome using Bowtie2 with default parameters. ncRNA genome annotations are used to identify known small RNAs such as miRNAs, tRNAs, snoRNAs, etc. Detection of novel small RNA loci is conducted on the remaining reads. miRNA detection is performed using miRDeep2, which uses thermodynamics to identify stable miRNA hairpin structures. The de novo detection of other small RNAs and piRNA clusters (e.g., SEQ ID NOS:5-22) is performed via an in-house script that first identifies transcripts by calculating the probability of getting an observed distribution of reads in the same region by chance. The novel and known loci together form a transcript assembly for which abundances are calculated and differentially expressed loci are identified, which is done using cufflinks with the—no-length-correction flag.

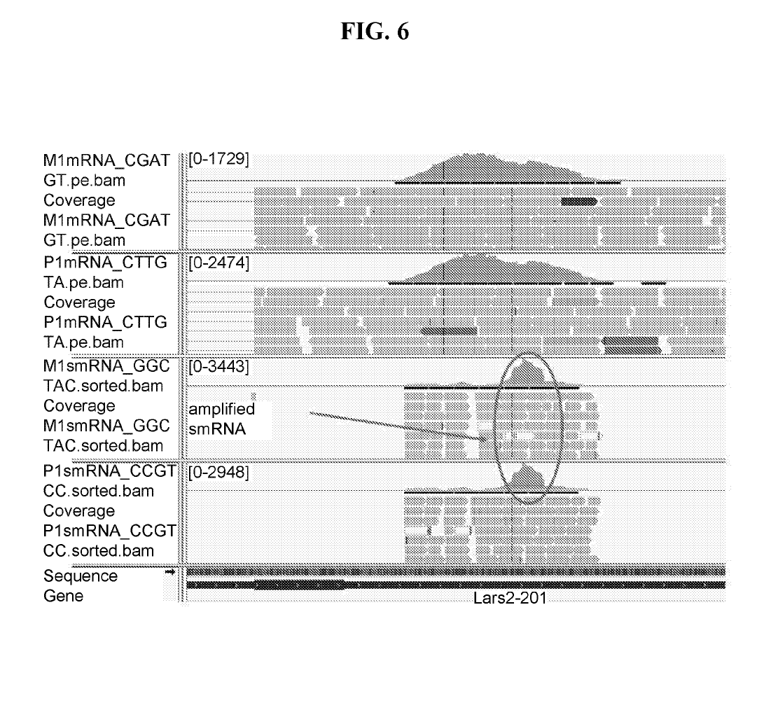

As shown in FIG. 6, it was found that there was a significant increase in small RNA abundance in MALAT1 ASO treated mice. In addition, many small RNAs with sequence complementarity to expressed protein coding loci exhibit amplification in the Malat1 ASO mice as compared to control mice. These results are consistent with MALAT1 impacting learning and memory consolidation through induction of small RNAs, which could be playing a regulatory role at their cognate coding loci, or through sequence-specific DNA methylation.

In further work, the DNA methylation level was examined in MALAT1 ASO treated mice. MALAT1 ASO #1 (SEQ ID NO: 2) was injected by IntraCerebroVentricular bolus (ICVB) injection (300 ug v. PBS control). After 2 weeks, the hippocampus was removed and the DNA was purified with a Qigen DNA Purification Kit. The DNA was then subject to bi-sulfite conversion with the Epi-Gnome kit from Epicentre (EpiGnome™ Methyl-Seq Kit). The converted DNA was sequenced with Illumina sequencing by synthesis technology. The reads were trimmed for adapter contamination, joined, and finally trimmed for low quality bases. Methylation calling was performed using Bismark with the default settings, in which the reads were aligned to converted versions of the genome and methylationed bases in the reads were inferred by identifying mismatches. The methylation calls were then extracted from the called reads to generate a single bp resolution methylation map of the genome. The bi-sulfite conversion error rate was estimated by counting the number of methylated bases in reads aligning to the Lambda control sequence. This error rate was used as a parameter in a maximum likelihood estimation (MLE) model that determined the methylation ratio at each cytosine in the genome with sequence coverage. Pooling the data across the biological replicates, the population methylation ratio at each site was determined assuming a normal distribution with random sampling. Paired t-tests were used to identify differentially methylated genes where the difference in mean methylation ratios between the conditions were statistically different than zero. Methylation profiles were calculated over the gene bodies and flanking regions using a 1 kb sliding window to smooth the data sufficiently for plotting.

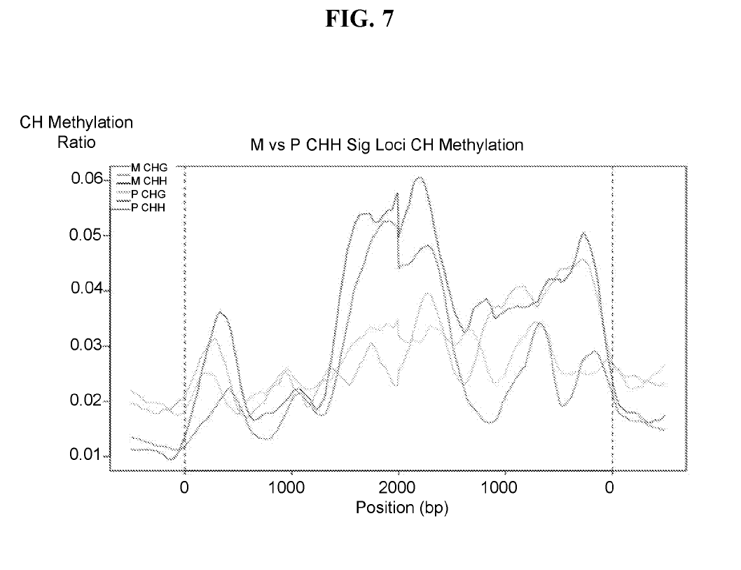

As shown in FIG. 7, it was found that DNA methylation was increased in MALAT1 ASO treated mice compared to controls. The 3′ end of the differentially methylated genes in the MALAT1 ASO mice (M) have increased methylation in the CHG and CHH contexts relative to the controls (P) (p=4.686e-09). These results suggest that the primary effect of MALAT1 in the learning process is increasing small RNAs and decreasing DNA methylation at specific gene loci. The changes in small RNA abundances and the DNA methylation are responsible for encoding the learning and memory event in fear conditioning. MALAT1 may play a direct role in regulating the epigenetic encoding of memories through small RNA directive sequence-specific DNA methytlation.

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method for improving memory or cognitive function in a subject, comprising:
administering to the subject a therapeutically effective dose of a composition that increases the expression of metastasis-associated lung adenocarcinoma transcript 1 (MALAT-1) long non-coding RNA in a cell of the nervous system of said subject,
wherein said administering is performed intracranially or wherein said administering is performed in a blood vessel that directly supplies blood to the brain of said subject;
wherein said composition comprises at least one of the following:
i) a compound that increases expression of MALAT-1 long non-coding RNA in said cell,
ii) a first nucleic acid sequence comprising said MALAT-1 long-coding RNA or a first expression vector encoding said first nucleic acid sequence; or
iii) a second nucleic acid sequence encoding at least one MALAT-1 derived piRNA sequence or a second expression vector encoding said second nucleic acid sequence; and
wherein at least one attribute of said memory or cognitive function is improved.

2. The method of claim 1, wherein said subject has a disorder in which diminished declarative memory is a symptom.

3. The method of claim 1, wherein said molecule is KCl or a DNA methyltransferase (DNMT) inhibitor.

4. The method of claim 3, wherein said DNMT inhibitor comprises RG108.

5. The method of claim 1, wherein said first and/or second expression vector comprises an adeno-associated virus (AAV), adenovirus, herpes simplex virus, lentivirus, or a DNA plasmid.

6. The method of claim 1, wherein said composition is administered to said subject by intracranial delivery through an intracranial access device.

7. The method of claim 6, further comprising the step of: implanting a pump outside said brain of said subject, wherein said pump is coupled to the proximal end of said intracranial access device.

8. The method of claim 6, wherein said intracranial access device comprises an intracranial catheter.

9. The method of claim 1, wherein said first and/or second nucleic acid molecule comprises a chemical modification that improves one or more or all of nuclease stability, decreased likelihood of triggering an innate immune response, lowering incidence of off-target effects, and improved pharmacodynamics relative to a non-modified nucleic acid.

10. The method of claim 9, wherein said at least one chemical modification selected from the group consisting of: phosphorothioate, boranophosphate, 4′-thio-ribose, locked nucleic acid, 2′-O-(2′-methoxyethyl), 2′-O-methyl, 2′-fluoro, 2′-deoxy-2′-fluoro-b-D-arabinonucleic acid, Morpholino nucleic acid analog, and Peptide nucleic acid analog.

11. The method of claim 1, wherein said first and/or second nucleic acid sequence is attached to, or inside of, a nanoparticle configured to cross the blood-brain barrier.

12. The method of claim 1, wherein said nanoparticle comprises a liposome.

13. The method of claim 1, wherein said composition is delivered to the nucleus basalis of Meynert, the cerebral cortex, or the hippocampus.

14. The method of claim 1, wherein said subject has Alzheimer's disease and/or age related memory decline.

15. The method of claim 1, wherein said subject has a memory impairment.

16. The method of claim 15, wherein said memory impairment is selected from the group consisting of: toxicant exposure, brain injury, age-associated memory impairment, mild cognitive impairment, epilepsy, mental retardation, and dementia resulting from a disease.

17. The method of claim 16, wherein said disease that results in dementia is selected from the group consisting of: Parkinson's disease, Alzheimer's disease, AIDS, head trauma, Huntington's disease, Pick's disease, Creutzfeldt-Jakob disease, post cardiac surgery, Downs Syndrome, Anterior Communicating Artery Syndrome, and symptoms of stroke.

18. The method of claim 1, wherein said subject has normal memory function that is desired to be enhanced.

19. The method of claim 1, wherein said administering is performed in a blood vessel that directly supplies blood to the brain of said subject.

20. A method of identifying a compound that modulates MALAT-1 long non-coding RNA in brain cells comprising:
a) contacting brain cells with a candidate agent, and
b) detecting the expression level of said MALAT-1 long non-coding RNA and/or a MALAT-1 derived piRNA,
wherein an increase or decrease in said expression level indicates that said candidate agent is a modulator of MALAT-1 long non-coding RNA in brain cells.

21. The method of claim 20, wherein said modulator increases said expression level, and wherein the method further comprises administering said modulator of MALAT-1 long non-coding RNA to the brain of a lab animal, and determining the impact of said modulator on memory or learning of said lab animal.

22. The method of claim 21, wherein said modulator is identified as increasing memory and/or learning in said animal.

23. The method of claim 20, wherein said brain cells are neurons or glial cells.

24. A method for treating alcoholism in a subject, comprising:
administering to the subject a therapeutically effective dose of a composition that comprises a MALAT-1 antisense,
wherein said administering is performed intracranially or wherein said administering is performed in a blood vessel that directly supplies blood to the brain of said subject; and
wherein at least one attribute of alcoholism in said subject is improved.


SEQUENCE LISTING (30 sequences)

SEQ ID NO: 1 - DNA, 6982 bp, Mus musculus
aggcattcaggcagcgagagcagagcagcgtagagcagcacagctgagctcgtgaggcaggagactcagcccgaggaaatcgcagataagtttttaattaaaaagattgagcagtaaaaagaattagaactctaaacttaagctaatagagtagcttatcgaaatattacttagtcttaataatctaagaagatcttaagagataacatgaaggcttatttaaacagtttgaaaaaggaaatgaggagaaaagtatttgtactgtataatggaggctgaccagagcagtttaggagattgtaaagggaggttttgtgaagttctaaaaggttctagtttgaaggtcggccttgtagattaaaacgaaggttacctaaatagaatctaagtggcatttaaaacagtaaagttgtagagaatagtttgaaaatgaggtgtagttttaaaagattgagaaaagtaggttaagttgacggccgttataaaaatccttcgactggcgcatgtacgtttgaaggcatgagttggaaacagggaagatggaagtgttaggctagccgggcgatggtggcgcacgcctttaatcctagcacttgggaggcagaggcaggcggatttctgagttcgaggccagcctggtctacagagtgagttccaggacagccagggctacacagagaaaccctgtcttgaaaaaacaaaaaggttaggctagtatttggagaaagaagattagaaaatggaagtgaaagacgaagaagacatacaggaaggtgaagaaaaagctgttagagaagataggaaaatagaagacaaagcatctttagaagacagaaaaggtacttaaaggcacaggtagtaggaagccgaagaatagaagatagaaagaagcaagatagaaaaacaaaatggaagttaagacaactttggatgccagcattcaagataggcaaagaagataagattgaggccaaaaggttggataagatataaagtcagaaggaaattatctttaaagccataagttcaaatttctgatggagcgagcagtttagaagagtctttagacagccacatacaagattgaagctagcaatcaaagctactaggactgaagtaaaaagttaaggcagaatgcctttgaagagttagaagaatattaaaagccttaacttgtagcttaattttgcttgatgacaaaaggacttttgataacagtttcaagattgtcagcattttgcattggacttgagctgaggtgcttttaaaatcctaacgactagcattggcagctgacccaggtctacacagaagtgcattcagtgaactaggaagacaggagcggcagacaggagtcccgaagccagtttggtgaagctaggaaggactgaggagccagcagcagcagtgcatggtgaagatagcccaggaaagagtgcggttcggtggaggaagctaggaagaaggagccatacggatgtggtggtgaagctgggaaagggttccaggatggtggagcgagagcgagttggtgatgaagctagctggcggcttggcttgtcaactgcgcggaggaggcgagcaggcattgtggagaggatagatagcggctcctagaccagcatgccagtgtgcaagaaaggctgcagggagagcatgcggtgcggtaacattccttgaggtcggcaacatggtggtggttttctgtaacttggatggtaacttgtttactttgtcttaatagttatgggggagttgtaggcttctgtgtaaagagatatatctggggctgtatgtaggcctttgcgggtgttgtaggtttttctttttcagggttatgtcctcttgcatcttgtcagaagcttttgagggctgactgccaaggcccagaaagaagaatggtagatggcaagttgtctttaaccgctcagaggggaatgaatggtagagccagcacaacctcccagttttgtaagacgttgtagtttgaacagatgacctaccacaagcctcactcctgtgtaggggaggtaattgggcaaagtgcttttgggggaatgggggcaaaatatattttgagttcttttccccttaggtctgtctagaatcctaaaggcagatgactcaagggaaccagaaaaaaggaaatccactctcaggataagcagagctcgccaggtttacagtttgtaggaagtagaggatggatgctagctttcacactgagtgtggaggagctggccatggcggaattgctggtagtttactctttccccctcccttaatgagatttgtaaaatcctaaacacttttacttgaaatatttgggagtggtcttaacagggaggagtgggtgggggaaacgttttttttctaagattttccacagatgctatagttgtgttgacacactgggttagagaaggcgtgtactgctatgctgttggcacgacaccttcagggactggagctgccttttgtccttggaagagttttcccagttgccgctgaagtcagcacagtgcggctttggttcacagtcacctcaggagaacctcaggagcttggctaggccagaggttgaagttaagttttacagcaccgtgatttaaaatatttcattaaaggggaggggtaaaacttagttggctgtggccttgtgtttgggtgggtgggggtgttaggtaattgtttagtttatgatttcagataatcataccagagaacttaaatatttggaaaaacaggaaatctcagctttcaagttggcaagtaactcccaatccagtttttgcttcttttttcctttttctttttttgaggcgggcagctaaggaaggttggttcctctgccggtccctcgaaagcgtagggcttgggggttggtctggtccactgggatgatgtgatgctacagtggggactcttctgaagctgttggatgaatatagattgtagtgtgtggttctcttttgaaatttttttcaggtgacttaatgtatcttaataactactataggaacaaaggaagtggctttaatgaccctgaaggaatttcttctggtgatagcttttatattatcaagtaagagatactatctcagttttgtataagcaagtctttttcctagtgtaggagaaatgattttccttgtgactaaacaagatgtaaaggtatgctttttttcttcttgtgcattgtatacttgtgtttatttgtaacttataatttaagaattatgataattcagcctgaatgtcttttagagggtgggcttttgttgatgagggaggggaaacctttttttttctgtagacctttttcagataacaccatctgagtcataaccagcctggcagtgtgatgacgtagatgcagagggagcagctccttggtgaatgagtgataagtaaaggcagaaaaaataatgtcatgtctccatggggaatgagcatgagccagagattgttcctactgatgaaaagctgcatatgcaaaaatttaagcaaatgaaagcaaccagtataaagttatggcaatacctttaaaagttatggcttatctaccaagctttatccacaaaagtaaagaattgatgaaaaacagtgaagatcaaatgttcatctcaaaactgcttttacaaaagcagaatagaaatgaagtgaaaatgctgcattaagcctggagtaaaaagaagctgagcttgttgagatgagtgggatcgagcggctgcgaggcggtgcagtgtgccaatgtttcgtttgcctcagacaggtttctcttcataagcagaagagttgcttcattccatctcggagcaggaaacagcagactgctgttgacagataagtgtaacttggatctgcagtattgcatgttagggatagataagtgccttttttctctttttccaaaaagacctgtagagctgttgaatgtttgcagctggcccctcttaggcagttcagaattttgagtagttttcccatccagcctcttaaaaattcctaagccttgcaccgatgggctttcatgatgggatagctaataggcttttgcatcgtaaacttcaacacaaaagcctacatgattaatgcctactttaattacattgcttacaagattaaggaatctttatcttgaagaccccatgaaagggatcattatgtgctgaaaattagatgttcatattgctaaaatttaaatgtgctccaatgtacttgtgcttaaaatcattaaattatacaaattaataaaatacttcactagagaatgtatgtatttagaaggctgtctccttatttaaataaagtcttgtttgttgtctgtagttagtgtgggcaattttggggggatgttcttctctaatcttttcagaaacttgacttcgaacacttaagtggaccagatcaggatttgagccagaagaccgaaattaactttaaggcaggaaagacaaattttattctccatgcagtgatgagcatttaataattgcaggcctggcatagaggccgtctaactaaggactaagtaccttaggcaggtgggagatgatggtcagagtaaaaggtaactacatattttgtttccagaaagtcaggggtctaatttgaccatggctaaacatctagggtaagacacttttcccccacatttccaaatatgcatgttgagtttaaatgcttacgatcatctcatccactttagccttttgtcacctcacttgagccacgagtggggtcaggcatgtgggtttaaagagttttcctttgcagagcctcatttcatccttcatggagctgctcaggactttgcatataagcgcttgcctctgtcttctgttctgctagtgagtgtgtgatgtgagaccttgcagtgagtttgtttttcctggaatgtggagggagggggggatggggcttacttgttctagctttttttttacagaccacacagaatgcaggtgtcttgacttcaggtcatgtctgttctttggcaagtaatatgtgcagtactgttccaatctgctgctattagaatgcattgtgacgcgactggagtatgattaaagaaagttgtgtttccccaagtgtttggagtagtggttgttggaggaaaagccatgagtaacaggctgagtgttgaggaaatggctctctgcagctttaagtaacccgtgtttgtgattggagccgagtccctttgctgtgctgccttaggtaaatgtttttgttcatttctggtgaggggggttgggagcactgaagcctttagtctcttccagattcaacttaaaatctgacaagaaataaatcagacaagcaacattcttgaagaaattttaactggcaagtggaaatgttttgaacagttccgtggtctttagtgcattatctttgtgtaggtgttctctctcccctcccttggtcttaattcttacatgcaggaacattgacaacagcagacatctatctattcaaggggccagagaatccagacccagtaaggaaaaatagcccatttactttaaatcgataagtgaagcagacatgccattttcagtgtggggattgggaagccctagttctttcagatgtacttcagactgtagaaggagcttccagttgaattgaaattcaccagtggacaaaatgaggacaacaggtgaacgagccttttcttgtttaagattagctactggtaatctagtgttgaatcctctccagcttcatgctggagcagctagcatgtgatgtaatgttggccttggggtggaggggtgaggtgggcgctaagcctttttttaagatttttcaggtacccctcactaaaggcactgaaggcttaatgtaggacagcggagccttcctgtgtggcaagaatcaagcaagcagtattgtatcgagaccaaagtggtatcatggtcggttttgattagcagtggggactaccctaccgtaacaccttgttggaattgaagcatccaaagaaaatacttgagaggccctgggcttgttttaacatctggaaaaaaggctgtttttatagcagcggttaccagcccaaacctcaagttgtgcttgcaggggagggaaaagggggaaagcgggcaaccagtttccccagcttttccagaatcctgttacaaggtctccccacaagtgatttctctgccacatcgccaccatgggcctttggcctaatcacagacccttcacccctcaccttgatgcagccagtagctggatccttgaggtcacgttgcatatcggtttcaaggtaaccatggtgccaaggtcctgtgggttgcaccagaaaaggccatcaattttccccttgcctgtaatttaacattaaaaccatagctaagatgttttatacatagcacctatgcagagtaaacaaaccagtatgggtatagtatgtttgataccagtgctgggtgggaatgtaggaagtcggatgaaaagcaagcctttgtaggaagttgttggggtgggattgcaaaaattctctgctaagactttttcaggtggacataacagacttggccaagctagcatcttagtggaagcagattcgtcagtagggttgtaaaggtttttcttttcctgagaaaacaaccttttgttttctcaggttttgctttttggcctttccctagctttaaaaaaaaaaaagcaaaagacgctggtggctggcactcctggtttccaggacggggttcaagtccctgcggtgtctttgcttgactcttatatcatgaggccattacatttttcttggagggttctaaaggctctgggtatggtagctgatatcactggaacactccccagcctcagtgttgaactcttgataattaactgcattgtctttcaggttatgcccaattcgtcttattacctctgagtcgacacacctcctactatttattgaatactttgattttatgaaataaaaactaaatatctctca

SEQ ID NO: 2 - DNA, 20 bp, ARTIFICIAL
  Synthetic
gccaggctggttatgactca

SEQ ID NO: 3 - DNA, 20 bp, ARTIFICIAL
  Synthetic
cggtgcaaggcttaggaatt

SEQ ID NO: 4 - DNA, 20 bp, ARTIFICIAL
  Synthetic
gggtcagctgccaatgctag

SEQ ID NO: 5 - DNA, 37 bp, ARTIFICIAL
  Synthetic
tgaaccccgtcctggaaaccaggagtgccagccacca

SEQ ID NO: 6 - DNA, 90 bp, ARTIFICIAL
  Synthetic
aagatgctagcttggccaagtctgttatgtccacctgaaaaagtcttagcagagaatttttgcaatcccaccccaacaacttcctacaaa

SEQ ID NO: 7 - DNA, 31 bp, ARTIFICIAL
  Synthetic
catccgacttcctacattcccacccagcact

SEQ ID NO: 8 - DNA, 48 bp, ARTIFICIAL
  Synthetic
cagccttttttccagatgttaaaacaagcccagggcctctcaagtatt

SEQ ID NO: 9 - DNA, 39 bp, ARTIFICIAL
  Synthetic
cacatcacacactcactagcagaacagaagacagaggca

SEQ ID NO: 10 - DNA, 35 bp, ARTIFICIAL
  Synthetic
gacaaaaggctaaagtggatgagatgatcgtaagc

SEQ ID NO: 11 - DNA, 34 bp, ARTIFICIAL
  Synthetic
tcggtcttctggctcaaatcctgatctggtccac

SEQ ID NO: 12 - DNA, 29 bp, ARTIFICIAL
  Synthetic
tgcccacactaactacagacaacaaacaa

SEQ ID NO: 13 - DNA, 84 bp, ARTIFICIAL
  Synthetic
aagaggctggatgggaaaactactcaaaattctgaactgcctaagaggggccagctgcaaacattcaacagctctacaggtctt

SEQ ID NO: 14 - DNA, 40 bp, ARTIFICIAL
  Synthetic
catcagtaggaacaatctctggctcatgctcattccccat

SEQ ID NO: 15 - DNA, 48 bp, ARTIFICIAL
  Synthetic
caccaaggagctgctccctctgcatctacgtcatcacactgccaggct

SEQ ID NO: 16 - DNA, 40 bp, ARTIFICIAL
  Synthetic
aaggcagctccagtccctgaaggtgtcgtgccaacagcat

SEQ ID NO: 17 - DNA, 87 bp, ARTIFICIAL
  Synthetic
catcctctacttcctacaaactgtaaacctggcgagctctgcttatcctgagagtggatttccttttttctggttcccttgagtcat

SEQ ID NO: 18 - DNA, 33 bp, ARTIFICIAL
  Synthetic
acacagaagcctacaactcccccataactatta

SEQ ID NO: 19 - DNA, 85 bp, ARTIFICIAL
  Synthetic
cactgctgctgctggctcctcagtccttcctagcttcaccaaactggcttcgggactcctgtctgccgctcctgtcttcctagtt

SEQ ID NO: 20 - DNA, 114 bp, ARTIFICIAL
  Synthetic
tgtcttctattttcctatcttctctaacagctttttcttcaccttcctgtatgtcttcttcgtctttcacttccattttctaatcttctttctccaaatactagcctaaccttt

SEQ ID NO: 21 - DNA, 46 bp, ARTIFICIAL
  Synthetic
tccctgtttccaactcatgccttcaaacgtacatgcgccagtcgaa

SEQ ID NO: 22 - DNA, 61 bp, ARTIFICIAL
  Synthetic
agtctcctgcctcacgagctcagctgtgctgctctacgctgctctgctctcgctgcctgaa

SEQ ID NO: 23 - DNA, 24 bp, ARTIFICIAL
  Synthetic
ggagtcctgttgatgttgccagta

SEQ ID NO: 24 - DNA, 24 bp, ARTIFICIAL
  Synthetic
gggacgcagcaactgacatttcta

SEQ ID NO: 25 - DNA, 22 bp, ARTIFICIAL
  Synthetic
aatggtgaagaccgtgtcagga

SEQ ID NO: 26 - DNA, 24 bp, ARTIFICIAL
  Synthetic
ttgatctgtctccgcttggagtgt

SEQ ID NO: 27 - DNA, 24 bp, ARTIFICIAL
  Synthetic
acgatctggcttcctcattctgct

SEQ ID NO: 28 - DNA, 24 bp, ARTIFICIAL
  Synthetic
aggttccctcagcatctctgcttt

SEQ ID NO: 29 - DNA, 19 bp, ARTIFICIAL
  Synthetic
agcgccttcaatcctcaag

SEQ ID NO: 30 - DNA, 20 bp, ARTIFICIAL
  Synthetic
tttggctgggataactcgtc